(19) United States
(12) Patent Application Publication
(10) Pub. No.: US 2016/0278163 A1
(43) Pub. Date: Sep. 22, 2016

(54) INHALER AND ATOMIZING ASSEMBLY THEREOF

(71) Applicant: SHENZHEN SMOORE TECHNOLOGY LIMITED, Shenzhen, Guangdong (CN)

(72) Inventor: Zhiping Chen, Shenzhen (CN)

(21) Appl. No.: 14/402,290

(22) PCT Filed: Oct. 10, 2014
(86) PCT No.: PCT/CN2014/088263

§ 371 Date: Nov. 19, 2014

(51) Int. Cl. H05B 3/00; A61M 11/04; A61M 15/06; A24F 47/00

(52) U.S. Cl. CPC H05B 3/0014; A24F 47/008; A61M 11/042; A61M 15/06

(57) ABSTRACT

An inhaler and an atomizing assembly thereof are provided. The liquid reservoir is sealed before use, such that the liquid stored in the liquid reservoir will not be volatile, thus prolonging the life use of the inhaler. In addition, since the liquid conducting element is located between the liquid reservoir and the liquid absorbing element, the liquid in the liquid reservoir can be more fluently directed to the liquid absorbing element by capillary force, thus increasing the atomizing efficiency.

### FIELD OF THE INVENTION

The present disclosure relates to devices used for delivering atomizing medium into the human body, and more particularly relates to an inhaler and an atomizing assembly thereof.

### BACKGROUND OF THE INVENTION

An inhaler is a device used for delivering atomized liquid into the human body, e.g. an electronic cigarette for replacing a traditional cigarette, or a medical inhaler used in the treatment of upper respiratory diseases and the like.

Taking the electronic cigarette as an example, when the conventional electronic cigarette is assembled, the internal liquid is in an unsealed state. Since the liquid will continue to be volatile during the time from assembly of the cigarette to the use by the user, the aromatic substances in the liquid may dissipate, resulting in a shorter service life of the conventional electronic cigarette.

### SUMMARY OF THE INVENTION

Accordingly, it is necessary to provide an inhaler and an atomizing assembly thereof to address these problems.

An atomizing assembly for an inhaler includes:

a housing having an airflow channel communicating with the outside and a closed liquid reservoir for storing liquid;

an atomizer removably received in the housing, wherein the atomizer includes:

a core having an atomizing cavity and a liquid absorbing hole communicated with the atomizing cavity, wherein the atomizing cavity is communicated with the airflow channel of the housing, and the core is provided with a piercing element at an end thereof;

an atomizing component including a liquid absorbing element and a heating element coupled to the liquid absorbing element, wherein the liquid absorbing element is received in the liquid absorbing hole, and the liquid conducting element is adjacent to the piercing element and connected to the liquid absorbing element; and

a liquid conducting element; and

wherein when the atomizer moves in the housing towards the liquid reservoir, the piercing element on the core is capable of piercing the liquid reservoir and extending inside the liquid reservoir, and the liquid conducting element is configured to guide the liquid flow to the liquid absorbing element after the piercing element extends inside the liquid reservoir.

In one embodiment, the core has a tubular shape. The atomizing cavity is located in the core. The atomizer further includes a fitting element sleeved on the core in sealing engagement with the housing.

In one embodiment, the core is provided with two limiting elements spaced apart from each other on an outer periphery thereof, and the sealing element is located between the two limiting elements.

In one embodiment, when the piercing element extends inside the liquid reservoir, one end of the liquid conducting element abuts one of the limiting elements and the other end of the liquid conducting element abuts the liquid reservoir.

In one embodiment, the atomizing cavity is provided with an air inlet and an air outlet. The atomizing assembly further includes an intake pipe located in the air inlet and an exhaust pipe located in the air outlet. The exhaust pipe is in communication with the airflow channel of the housing.

In one embodiment, the atomizing assembly further includes a sealing element sleeved on the exhaust pipe. When the piercing element extends inside the liquid reservoir, the liquid reservoir is isolated from the airflow channel via the sealing element.

In one embodiment, the core is provided with a stepped portion therein, and the exhaust pipe is located on the stepped portion.

In one embodiment, the atomizing assembly further includes an insulating element located in the air inlet configured to isolate the core from the intake pipe.

An inhaler includes a power assembly and any one atomizing assembly described above, wherein the power assembly is configured to be fixed to the atomizing assembly. After the power assembly is fixed to the atomizing assembly, the power assembly abuts the atomizer such that the piercing element on the core pierces the liquid reservoir and extends inside the liquid reservoir, and the liquid conducting element guides the liquid flow to the liquid absorbing element, and the power assembly is electrically connected to the atomizing component.

In one embodiment, the housing includes a first tubular structure and a second tubular structure located inside the first tubular structure. The airflow channel is formed in the inside of the second tubular structure, the liquid reservoir is formed between the first tubular structure and the second tubular structure.

In one embodiment, the inhaler further includes a sealing sheet located at an end of the first tubular structure and second tubular structure configured to seal the liquid reservoir, wherein the sealing sheet has an annular shape and defines an opening in communication with the airflow channel.

In one embodiment, the liquid conducting element abuts the sealing sheet after the power assembly is fixed to the atomizing assembly.

In one embodiment, the housing further comprises a connecting element configured to connect the power assembly. The connecting element and the liquid reservoir form a receiving cavity therebetween, and the atomizer is removably received in the receiving cavity before the power assembly is fixed to the atomizing assembly.

In the present inhaler and atomizing assembly thereof, the liquid reservoir is sealed before use such that the liquid stored in the liquid reservoir will not be volatile, thus prolonging the life use of the inhaler. In addition, since the liquid conducting element is located between the liquid reservoir and the liquid absorbing element, the liquid in the liquid reservoir can be more fluently directed to the liquid absorbing element by capillary force, thus increasing the atomizing efficiency.

### BRIEF DESCRIPTION OF THE DRAWINGS

FIG. 1 is a perspective view of an inhaler according to one embodiment;

FIG. 2 is a cross-sectional view of the inhaler of FIG. 1 during use, wherein a power assembly is fixed to an atomizing assembly;

FIG. 3 is an enlarged sectional view in correspondence with the enlarged portion of FIG. 2;

FIG. 4 is a cross-sectional view of the atomizing assembly of the inhaler according to another embodiment illustrating a state before it is fixed to the power assembly;

FIG. 5 is an enlarged sectional view in correspondence with the enlarged portion of FIG. 4;

FIG. 6 is a cross-sectional view of an atomizer of the atomizing assembly according to an embodiment;

FIG. 7 is a perspective view of a core of the atomizer according to an embodiment;

FIG. 8 is a partial, cross-sectional view of a power assembly of the inhaler according to an embodiment.

### DETAILED DESCRIPTION OF THE EMBODIMENTS

Embodiments of the invention are described more fully hereinafter with reference to the accompanying drawings. The various embodiments of the invention may, however, be embodied in many different forms and should not be construed as limited to the embodiments set forth herein. Rather, these embodiments are provided so that this disclosure will be thorough and complete, and will fully convey the scope of the invention to those skilled in the art.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

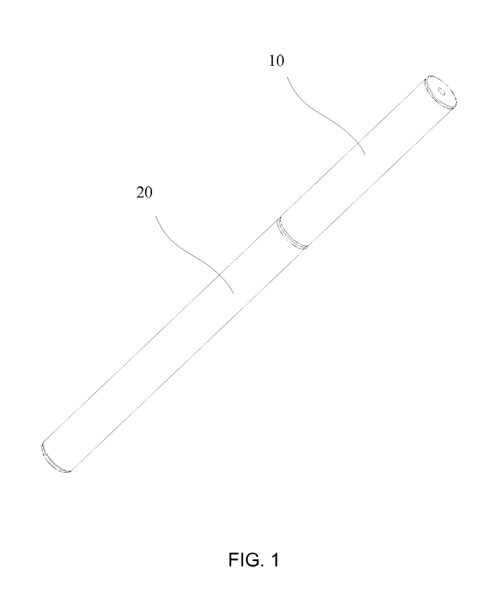

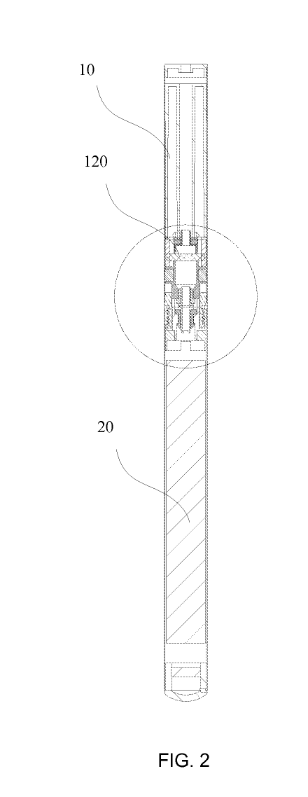

Referring to FIG. 1 and FIG. 2, an embodiment of an inhaler includes an atomizing assembly 10 and a power assembly 20. During use of the inhaler, the atomizing assembly 10 is fixed to the power assembly 20, and the power assembly 20 can provide power for the atomizing assembly 10 to atomize the liquid stored in the atomizing assembly 10. The user can inhale the atomized liquid from an end of the atomizing assembly 10, e.g. the top end shown in FIG. 2. The inhaler according to the present embodiment can be an electronic cigarette, as well as a medical inhaler, or another instrument.

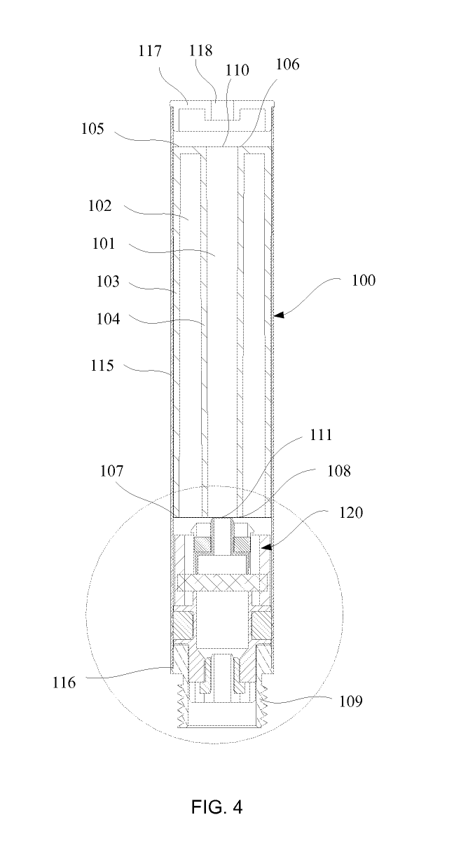

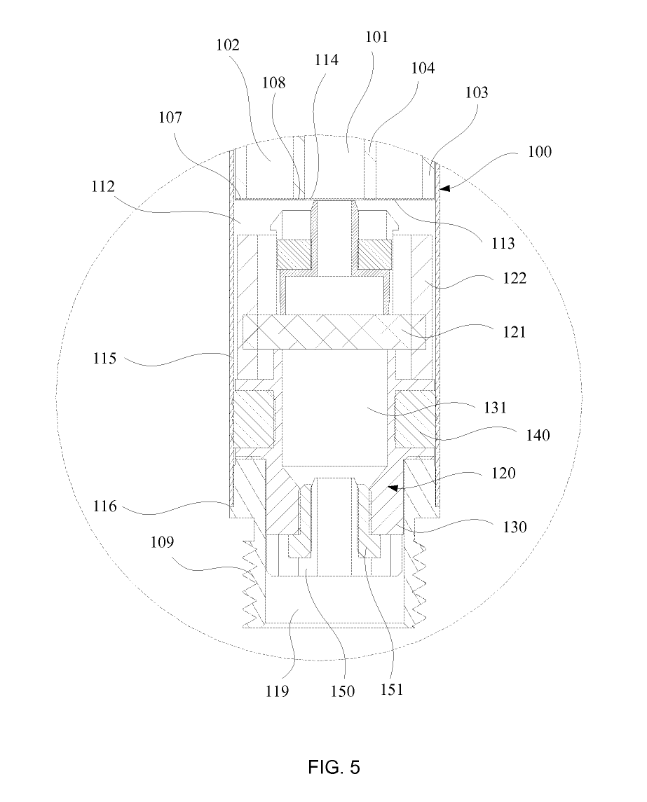

Referring to FIG. 4 and FIG. 5, the atomizing assembly 10 is shown in a state before it is connected to the power assembly 20.

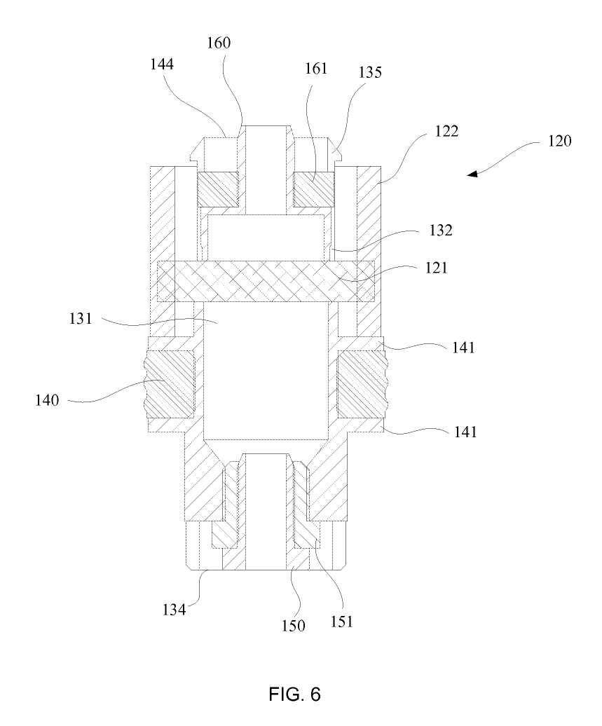

The atomizing assembly 10 includes a housing 100 and an atomizer 120 removably received in the housing 100. FIG. 6 shows a cross-section of the atomizer 120. The housing 100 has a substantially tubular shape. The atomizer 120 can move axially along the housing 100.

The housing 100 internally defines an airflow channel 101 to communicate with the outside and a closed liquid reservoir 102 for storing liquid. When the atomizer 120 is used as a component of an electronic cigarette, the liquid can be electronic cigarette liquid, which can be atomized and flow out from the airflow channel 101 as a smoke, thus giving the user a feeling of smoking tobacco. Alternatively, the atomizing assembly 10 can also be used as components of other equipment, thus the stored liquid may vary depending on the application environment.

In one embodiment, the housing 100 includes a first tubular structure 103 and a second tubular structure 104 located inside the first tubular structure 103. The airflow channel 101 is formed in the inside of the second tubular structure 104. The liquid reservoir 102 is formed between the second tubular structure 104 and the first tubular structure 103.

In other words, in the illustrated embodiment shown in FIG. 4 and FIG. 5, the liquid reservoir 102 surrounds the airflow channel 101. In alternative embodiments, the airflow channel 101 and the liquid reservoir 102 can be structurally interchangeable, i.e., the airflow channel 101 can surround the liquid reservoir 102. In that case, the structure of the atomizer 120 may be adjusted accordingly, which will be further illustrated with the description of the atomizer 120.

The first tubular structure 103 and the second tubular structure 104 can be integrally formed. A first end 105 (top end shown in FIG. 4) of the first tubular structure 103 and a first end 106 (top end shown in FIG. 4) of the second tubular structure 104 are in the same level. A second end 107 (bottom end shown in FIG. 4) of the first tubular structure 103 and a second end 108 (bottom end shown in FIG. 4) of the second tubular structure 104 are in the same level. In one embodiment, the first end 105 of the first tubular structure 103 and the first end 106 of the second tubular structure 104 are connected to form a closed space, and the first end 106 of the second tubular structure 104 is opened to form an exit 110 of the airflow channel 101. The second end 108 of the second tubular structure 104 forms an entrance 111 of the airflow channel 101.

The second end 107 of the first tubular structure 103 and the second end 108 of the second tubular structure 104 are provided with a sealing sheet 113 configured to seal the liquid reservoir 102. The sealing sheet 113 has an annular shape and defines an opening 114 communicated with the airflow channel 101, thus allowing the entrance 111 of the airflow channel 101 to be in an open state. The sealing sheet 113 can be made of aluminum foil and other materials. The sealing sheet 113 can become opened when subjected to external force by a sharp object, while it can maintain sealing under a large force area.

In alternative embodiments, the sealing sheet 113 can seal both the space between the second ends 107, 108 of the first and the second tubular structures 103, 104 and the second end 108 of the second tubular structure 104. In other words, the sealing sheet 113 may have a circular shape rather than an annular shape such that the liquid reservoir 102 as well as the entrance 111 of the airflow channel 101 are sealed. In this case, the entrance 111 of the airflow channel 101 can be opened during the connection of the atomizing assembly 10 to the power assembly 20 when in use.

In one embodiment, the housing 100 further includes a connecting element 109 configured to connect the power assembly 20. The connecting element 109 and the liquid reservoir 102 form a receiving cavity 112 therebetween, and the atomizer 120 is removably received in the receiving cavity 112 before the power assembly 20 is fixed to the atomizing assembly 10.

The connecting element 109 is hollow and defines a passage 119 therein, such that when connecting the power assembly 20, the components of the power assembly 20 can extend through the passage 119 and abut the atomizer 120. Accordingly, the atomizer 120 can move towards the liquid reservoir 102 during the connection of the power assembly 20 and the atomizing assembly 10.

Specifically, the housing 100 further includes an outer housing 115 which is positioned on the outside of the first tubular structure 103, and the second end 116 of the outer housing 115 (bottom end shown in FIG. 4) extends beyond the second ends 107, 108 of the first and second tubular structures 104. The connecting element 109 is located at the second end 116 of the outer housing 115. The receiving cavity 112 is substantially bordered by the sealing sheet 113, outer housing 115, and the connecting element 109. The connecting element 109 is used to prevent the atomizer 120 from detaching from the receiving cavity 112. In some embodiments, the connecting element 109 may be a thread or latching means and the like, so as to achieve the fixed connection between the atomizing assembly 10 and the power source 20.

In the illustrated embodiment shown in FIG. 4 and FIG. 5, the outer housing 115 and the first tubular structure 103 are separate and independent components. In alternative embodiments, the outer housing 115 and the first tubular structure 103 can be integrally formed, and a stepped portion for placing the sealing sheet 113 is provided at a place where the outer housing 115 is aligned with the second end 108 of the second tubular structure 104.

It should be understood that the outer housing 115 and the first tubular structure 103 can be at least partially transparent, thus facilitating the user to observe the liquid capacity in the liquid reservoir 102.

Referring to FIG. 4, in alternative embodiments, the first ends 105, 106 of the first and the second tubular structures 103, 104 can be provided with a nozzle 117 having an air suction hole 118 configured to communicate with the airflow channel 101.

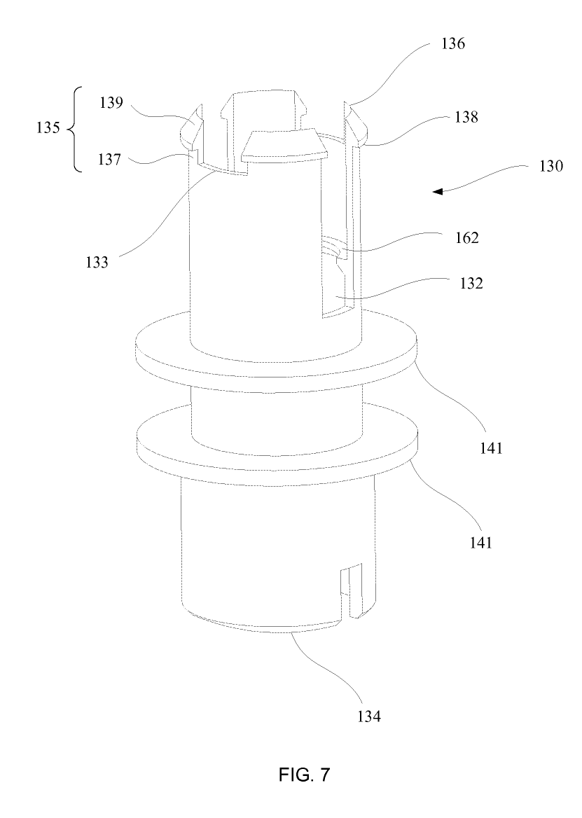

Referring to FIG. 6 and FIG. 7, the atomizer 120 includes a core 130, an atomizing component, and a liquid conducting element 122.

The core 130 defines an atomizing cavity 131 therein and a liquid absorbing hole 132 in communication with the atomizing cavity 131. The atomizing cavity 131 is in communication with the airflow channel 101 of the housing 100. The core 130 is provided with a piercing element 135 at a first end 133 thereof (the top end shown in FIG. 6 and FIG. 7). When the atomizer 120 moves towards the liquid reservoir 102 in the housing 100, the piercing element 135 on the core 130 is configured to pierce the liquid reservoir 102 and extend inside the liquid reservoir 102.

The atomizing component includes a liquid absorbing element 121 and a heating element (not shown) coupled to the liquid absorbing element 121. The liquid absorbing element 121 extends through the liquid absorbing hole 132. The number of liquid absorbing holes 132 is two, and two liquid absorbing holes 132 are positioned symmetrically on the core 130. The liquid absorbing element 121 has a rod-like shape and extends through the inside of the liquid absorbing hole 132. It should be understood that there may be multiple liquid absorbing holes 132, and the shape of the liquid absorbing element 121 may not be limited to rod-like, for example, it may be shaped as stand constituted by multiple rods intersected in middle portions.

When the power assembly 20 is fixed to the atomizing assembly 10, the liquid absorbing element 121 is configured to absorb the liquid in the liquid reservoir 102. The heating element is electrically coupled to the power assembly 20, thus heating and atomizing the liquid in the liquid absorbing element in an electricity-to-heat manner. The heating element can be heating wire wound on the liquid absorbing element 121 or it may be heating wire extending through the liquid absorbing element 121 to achieve the coupling of the heating element with the liquid absorbing element 121. The liquid absorbing element 121 can be made of liquid-absorbent cotton, fibers, or the like.

In one embodiment, the core 130 is tubular to match the corresponding structure of the housing 100 shown in FIG. 4 and FIG. 5. The atomizing cavity 131 is located inside the core 130 such that it can communicate with the airflow channel 101.

In alternative embodiments, when the airflow channel 101 is positioned between the first tubular structure 103 and the second tubular structure 104, the atomizing cavity 131 can be located between the core 130 and the outer housing 115 so as to achieve communication between the atomizing cavity 131 and the airflow channel 101.

Referring to FIG. 7, the piercing element 135 includes a base portion 137 and a hook portion 139 extending outwardly from the base portion 137. At least one cross-sectional area of the hook portion 139 is greater than that of the base portion 137.

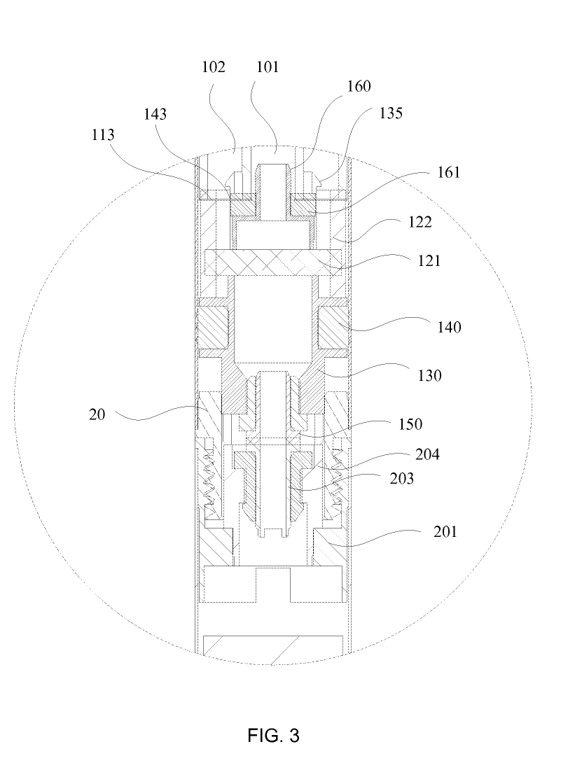

Specifically, the hook portion 139 may be tapered or wedge-shaped. The hook portion 139 includes opposed smaller end 136 and bigger end 138. The smaller end 136 faces the liquid reservoir 102 and the bigger end 138 is connected to the base portion 137. The smaller end 136 has a sharp shape, e.g., round point or line, so as to facilitate puncturing of the sealing sheet 113. The base portion 137 has a sheet-like shape. In the illustrated embodiment shown in FIG. 7, there is a size difference between the base portion 137 and the hook portion 139, and the entire cross-sectional area of the base portion 137 is equal. The bigger end 138 of the hook portion 139 has an apparently larger cross-sectional area than that of the base portion 137, and the cross-sectional area of the hook portion 138 is gradually decreasing in a direction from the bigger end 138 to the smaller end 136. Referring also to FIG. 3, when the power assembly 20 is fixed to the atomizing assembly 10, the atomizer 120 will be forced by the power assembly 20 to move in the receiving cavity 112 towards the liquid reservoir 102, such that the sealing sheet 113 is pierced by the piercing element 135 and forms a through hole 143, and the hook portion 139 of the piercing element 135 is entirely received in the liquid reservoir 102, at least part of the base portion 137 of the piercing element 135 is located in the liquid reservoir 102. The shapes of the base portion 137 and the hook portion 139 of the piercing element 135 determine that the area of the through hole 143 is greater than the cross-sectional area of the base portion 137. Therefore, the liquid in the liquid reservoir 102 can freely flow through the through hole 143 due to the size difference between the through hole 143 and the base portion 137, thus avoiding the liquid from being still unable to successfully outflow due to the influence of surface tension, and the problem of poor circulation of the liquid is overcome.

Furthermore, the liquid conducting element 122 is disposed adjacent to the piercing element 135 and is connected to the liquid absorbing element 121. When the atomizer 120 moves in the housing 100 towards the liquid reservoir 102, the liquid conducting element 122 can guide the liquid to flow to the liquid absorbing element 121. The liquid conducting element 122 can be made of liquid-conducting cotton or other materials having a microporous structure, preferably made of soft materials. Since the liquid conducting element 122 is disposed adjacent to the piercing element 135, when the liquid reservoir is opened by the piercing element 135, the liquid in the liquid reservoir 102 can be directed to the liquid absorbing element 121 by the liquid conducting element 122 using capillary force, thereby further reducing the negative effects of surface tension and other factors, making the liquid in the liquid reservoir flow more smoothly to the liquid absorbing element 121 facilitating atomization.

The term “disposed adjacent” can be understood that by placing the piercing element 135 adjacent to the liquid conducting element 122, the following objectives are achieved: after the piercing element 135 opens the sealing sheet 113 of the liquid reservoir 102, the liquid conducting element 122 can guide the liquid to flow to the liquid absorbing element 121 using capillary force. Therefore, “adjacent” means a certain distance range where the liquid conducting element 122 is able to cast the capillary force, and the distance range is at least related to the type of material of the liquid conducting element 122 and the type of liquid inside the liquid reservoir 102. In alternative embodiments, the liquid conducting element 122 can be connected to the piercing element 135 directly such that when the sealing sheet 113 is penetrated by the piercing element 135, the liquid conducting element 122 can pass the through hole 143 and connect to the liquid reservoir 102 directly. In alternative embodiments, the liquid conducting element 122 can be spaced a certain distance apart from the piercing element 135. After the piercing element 135 penetrates the sealing sheet 113 and partially enters the liquid reservoir 102, the liquid conducting element 122 may be deformed by the abutting of the sealing sheet 113 such that the liquid conducting element 122 can extend towards the through hole 143 and the capillary force of the liquid conducting element 122 can be achieved. Preferably, it can be predetermined that, before connecting between the power assembly 20 and the atomizing assembly 10, the distance between the liquid conducting element 122 and the sealing sheet 113 is less than the moving distance of the piercing element 135 after the connection between the power assembly 20 and the atomizing assembly 10, such that after the power assembly 20 is fixed to the atomizing assembly 10, the liquid conducting element 122 can be deformed by the abutting of the sealing sheet 113.

In some embodiments, the piercing elements 135 can be plural and are positioned on the first end 133 of the core 130 spaced apart from each other.

Referring to FIG. 6, the atomizer 120 further includes a fitting element 140, which is sleeved on the core 130. The fitting element 140 can be made of soft material having sealing properties, such as silicone and the like. The fitting element 140 can pre-fix the atomizer 120 in the housing 100 (referring to FIG. 5) and form a sealing engagement between the atomizer 120 and the housing 100, thus preventing leakage of the liquid after the liquid reservoir 102 is opened by the piercing element 135. When the atomizer 120 is pushed by the power assembly 20, it can overcome the friction between the atomizer 120 and the housing 100 so as to move towards the liquid reservoir 102. It should be understood that, if the liquid reservoir 102 is formed inside the second tubular structure 104, the fitting element 140 may not have to form a sealing engagement to the housing 100.

Still further, in alternative embodiments, the core 130 is provided with two limiting elements 141 spaced apart from each other on an outer periphery thereof, and the fitting element 140 is located between the two limiting elements 141.

After the piercing element 135 extends inside the liquid reservoir 102, one end of the liquid conducting element 122 abuts one of the limiting elements 141 and the other end of the liquid conducting element 122 abuts the liquid reservoir 102. Specifically, one end of the liquid conducting element 122 abuts one limiting element 141 which is close to the first end 133 of the core 130 and the other end of the liquid conducting element 122 abuts the sealing sheet 113 of the liquid reservoir 102.

Still further, referring to FIG. 6, the atomizing cavity 131 of the core 130 is provided with an air inlet 134 and an air outlet 144. The atomizing assembly 10 further includes an intake pipe 150 located in the air inlet 134 and an exhaust pipe 160 located in the air outlet 144. The exhaust pipe 160 is in communication with the airflow channel 101 of the housing 100.

In some embodiments, the atomizing assembly 10 further includes a sealing element 161 sleeved on the exhaust pipe 160. When the piercing element 135 extends inside the liquid reservoir 102, the liquid reservoir 102 is isolated from the airflow channel 101 via the sealing element 161 (see FIG. 3).

Moreover, referring to FIG. 7, the core 130 is provided with a stepped portion 162 therein, and the exhaust pipe 160 is located on the stepped portion 162.

Moreover, the atomizing assembly 10 further includes an insulating element 151 which is located in the air inlet 134 and configured to isolate the core 130 from the intake pipe 150. The intake pipe 150, as well as the core 130, can be made of conducting materials. When two electrodes of the heating wire of the heating element are electrically coupled to the power assembly 20, one of the electrodes may be located between the insulating element 151 and the intake pipe 150, the other electrode is located between the insulating element 151 and the core 130, such that the intake pipe 150 and the core 130 form two electrodes electrically coupled to the corresponding structure of the power assembly 20.

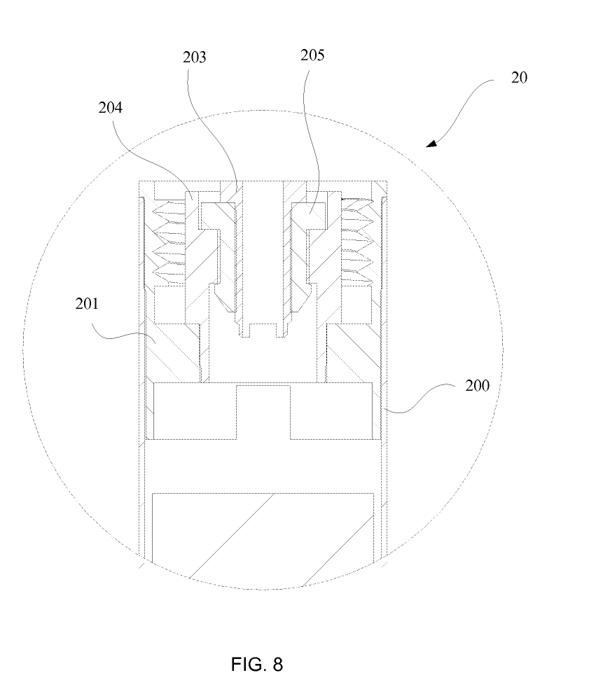

Referring to FIG. 8, the power assembly 20 includes a housing 200, a connecting element 201 located in the housing 200, and two electrode portions 203, 204 located in the housing 200 are insulated from each other. Similar to the insulating element 151 located between the intake pipe 150 and the core 130, the two electrode portions 203, 204 can be insulated by an insulating element 205. The connecting element 201 is configured to cooperate with the connecting element 109 to form a fixed connection between the power assembly 20 and the atomizing assembly 10. In the illustrated embodiment shown in FIG. 8, the connecting element 201 is a thread which can be threadly engaged with the connecting element 109 of the atomizing assembly 10 shown in FIG. 3. During the process of thread engagement, the two electrode portions 203, 204 of the power assembly 20 abut the intake pipe 150 and the core 130, respectively, thus overcoming the friction between the fitting element 140 and the housing 100 and forcing the atomizer 120 to move towards the liquid reservoir 102. Therefore, the piercing element 135 of the core 130 finally can penetrate the sealing sheet 113 and open the liquid reservoir 102 such that the liquid is directed by the liquid conducting element 122 to flow to the liquid absorbing element 121 while the connection between the power assembly 20 and the atomizing assembly 10 is completed.

It should be understood that the power assembly 20 may also include switches, simulate lightings, and other components, which will not be described in further detail.

In the present invention, the liquid reservoir 102 is sealed before use such that the liquid stored in the liquid reservoir 102 will not be volatile, thus prolonging the life use of the inhaler. In addition, since the piercing element 135 has a special structure employed when the liquid reservoir 102 is opened, the liquid in the liquid reservoir 102 can flow fluently to the liquid absorbing element 121, thus increasing the atomizing efficiency. Furthermore, since the liquid conducting element 122 is located between the liquid reservoir 102 and the liquid absorbing element 121, the liquid in the liquid reservoir 102 can be more fluently directed to the liquid absorbing element 121 by capillary force, thus further increasing the atomizing efficiency.

Although the present invention has been described with reference to the embodiments thereof and the best modes for carrying out the present invention, it is apparent to those skilled in the art that a variety of modifications and changes may be made without departing from the scope of the present invention, which is intended to be defined by the appended claims.

What is claimed is:

1. An atomizing assembly for an inhaler, comprising:
a housing having an airflow channel for communicating with an outside and a closed liquid reservoir for storing liquid;
an atomizer removably received in the housing, wherein the atomizer comprises:
a core having an atomizing cavity and a liquid absorbing hole in communication with the atomizing cavity, wherein the atomizing cavity is in communication with the airflow channel of the housing, and the core is provided with a piercing element at an end thereof;
an atomizing component comprising a liquid absorbing element and a heating element coupled to the liquid absorbing element, wherein the liquid absorbing element is received in the liquid absorbing hole; and
a liquid conducting element adjacent to the piercing element and connected to the liquid absorbing element;
wherein when the atomizer moves in the housing towards the liquid reservoir, the piercing element on the core pierces the liquid reservoir and extends inside the liquid reservoir, and the liquid conducting element guides the liquid to the liquid absorbing element after the piercing element extends inside the liquid reservoir.

2. The atomizing assembly for the inhaler according to claim 1, wherein the core has a tubular shape, the atomizing cavity is located in the core,
and the atomizer further comprises a fitting element sleeved on the core in sealing engagement with the housing.

3. The atomizing assembly for the inhaler according to claim 2, wherein the core is provided with two limiting elements spaced apart from each other on an outer periphery thereof, and the fitting element is located between the two limiting elements.

4. The atomizing assembly for the inhaler according to claim 3, wherein when the piercing element extends inside the liquid reservoir, one end of the liquid conducting element abuts one of the limiting elements, and the other end of the liquid conducting element abuts the liquid reservoir.

5. The atomizing assembly for the inhaler according to claim 2, wherein the atomizing cavity is provided with an air inlet and an air outlet, the atomizing assembly further comprises an intake pipe located in the air inlet and an exhaust pipe located in the air outlet, the exhaust pipe is in communication with the airflow channel of the housing.

6. The atomizing assembly for the inhaler according to claim 5, further comprising a sealing element sleeved on the exhaust pipe, when the piercing element extends inside the liquid reservoir, the liquid reservoir is isolated from the airflow channel via the sealing element.

7. The atomizing assembly for the inhaler according to claim 6, wherein the core is provided with a stepped portion therein, and the exhaust pipe is located on the stepped portion.

8. The atomizing assembly for the inhaler according to claim 5, further comprising an insulating element located in the air inlet configured to isolate the core from the intake pipe.

9. An inhaler, comprising a power assembly and an atomizing assembly, the atomizing assembly further comprising:
a housing having an airflow channel for communicating with an outside and a closed liquid reservoir for storing liquid;
an atomizer removably received in the housing, wherein the atomizer comprises:
a core having an atomizing cavity and a liquid absorbing hole in communication with the atomizing cavity, wherein the atomizing cavity is in communication with the airflow channel of the housing, and the core is provided with a piercing element at an end thereof;
an atomizing component comprising a liquid absorbing element and a heating element coupled to the liquid absorbing element, wherein the liquid absorbing element is received in the liquid absorbing hole; and
a liquid conducting element adjacent to the piercing element and connected to the liquid absorbing element;
wherein when the atomizer moves in the housing towards the liquid reservoir, the piercing element on the core pierces the liquid reservoir and extends inside the liquid reservoir, and the liquid conducting element guides the liquid to the liquid absorbing element after the piercing element extends inside the liquid reservoir; and
wherein the power assembly is fixed to the atomizing assembly, the power assembly abuts the atomizer such that the piercing element on the core pierces the liquid reservoir and extends inside the liquid reservoir, and the liquid conducting element guides the liquid flow to the liquid absorbing element after the piercing element extends inside the liquid reservoir; and
wherein the power assembly is electrically connected to the atomizing component.

10. The inhaler according to claim 9, wherein the housing comprises a first tubular structure and a second tubular structure located inside the first tubular structure, the airflow channel is formed in the inside of the second tubular structure, and the liquid reservoir is formed between the first tubular structure and the second tubular structure.

11. The inhaler according to claim 10, further comprising a sealing sheet located at an end of the first tubular structure and the second tubular structure configured to seal the liquid reservoir, wherein the sealing sheet has an annular shape and defines an opening in communication with the airflow channel.

12. The inhaler according to claim 11, wherein the liquid conducting element abuts the sealing sheet after the power assembly is fixed to the atomizing assembly.

13. The inhaler according to claim 9, wherein the housing further comprises a connecting element configured to connect the power assembly to the atomizing assembly, the connecting element and the liquid reservoir form a receiving cavity therebetween, and the atomizer is removably received in the receiving cavity before the power assembly is connected to the atomizing assembly.
